(19) United States
(12) Patent Application Publication
(10) Pub. No.: US 2025/0213514 A1
(43) Pub. Date: Jul. 3, 2025

(54) STABLE, LIQUID PHARMACEUTICAL COMPOSITIONS COMPRISING MELPHALAN

(71) Applicant: GOOD HEALTH, LLC, Princeton, NJ (US)

(72) Inventor: Basavaraj SIDDALINGAPPA, Gujarat (IN)

(21) Appl. No.: 18/881,146

(22) PCT Filed: Jul. 6, 2023
(86) PCT No.: PCT/US2023/069700

§ 371 Date: Jan. 3, 2025

Related U.S. Application Data
(60) Provisional application No. 63/358,649, filed on Jul. 6, 2022.

(51) Int. Cl. A61K 31/198; A61K 9/08; A61K 47/10; A61K 47/18; A61K 47/40

(52) U.S. Cl. CPC A61K 31/198; A61K 9/08; A61K 47/10; A61K 47/183; A61K 47/40

(57) ABSTRACT

The invention is directed to stable, liquid pharmaceutical compositions comprising melphalan, at least one cyclodextrin, at least one non-aqueous solvent, water and/or at least one aqueous buffer, at least one antioxidant, optionally, at least one chelating agent, and, optionally, at least one inorganic salt. The invention is also directed to the use of the compositions of the invention for the treatment of cancers, their preparation, and dosage forms containing them.

### CROSS-REFERENCE TO RELATED APPLICATIONS

This application claims priority to U.S. Provisional Application No. 63/358,649, filed Jul. 6, 2022, which is incorporated herein by reference in its entirety.

### FIELD OF THE INVENTION

This invention relates to stable, liquid pharmaceutical compositions of melphalan, methods for their use and preparation, and dosage forms containing them.

### BACKGROUND OF THE INVENTION

Drugs intended for intravenous injection are required to have adequate solubility in water or aqueous physiologically acceptable buffers. The injectable solutions must demonstrate adequate stability during storage, preferably under room temperature conditions.

Lack of solubility in aqueous fluids can lead to making injectable formulations in non-aqueous organic solvents. Such products need dilution with physiologically compatible diluents. Even if the formulations are made with non-aqueous solvents, stability of such formulations cannot be guaranteed.

Organic solvents are often used to formulate injectable formulations of drugs. The injectable formulation made with higher amounts of organic solvents are usually diluted with isotonic diluents such as sodium chloride and dextrose solutions prior to injection.

Non-aqueous products, which are completely devoid of water, also sometimes have limited stability as-is and also after diluting with aqueous diluents.

To avoid stability issues, injectable drugs are typically lyophilized to obtain fast dissolving sterile cakes that are reconstituted with diluent to get injectable solutions.

Certain drugs, even after lyophilization, require organic solvents to dissolve the lyophilized cake, and, even after reconstitution, the resulting solutions may not be stable for an extended period. Rather, such solutions must be stored under refrigerated conditions, if the solutions are not to be used immediately.

Melphalan, sold under the brand name Alkeran® among others, is a chemotherapy medication used to treat multiple myeloma, ovarian cancer, melanoma, and AL amyloidosis. It is also marketed as Evomela®, which has been approved for use as a high-dose conditioning treatment prior to hematopoietic progenitor (stem) cell transplantation in multiple myeloma (MM) patients, and the palliative treatment of MM patients for whom oral therapy is not appropriate.

Melphalan is a DNA alkylating agent, and it acts by chemically altering the DNA nucleotide guanine through alkylation, and causes linkages between strands of DNA. This chemical alteration inhibits DNA synthesis and RNA synthesis, functions necessary for cells to survive. These changes cause cytotoxicity in both dividing and non-dividing tumor cells. Melphalan has the following chemical structure:

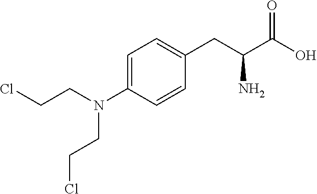 [SMILES: N[C@@H](Cc1ccc(N(CCCl)CCCl)cc1)C(=O)O]

Melphalan is soluble in propylene glycol and dilute mineral acids; slightly soluble in ethanol, methanol; and practically insoluble in water, chloroform, and ether.

Melphalan HCl for intravenous injection, marketed as Alkeran®, is supplied as a lyophilized cake, consisting of melphalan hydrochloride, which is equivalent to 50 mg melphalan, and 20 mg povidone and includes a sterile diluent. Each vial of the sterile diluent contains 6 mL of propylene glycol, 0.52 mL of ethanol (96%), 0.2 g of sodium citrate, and water q.s. up to a total of 10 mL. The lyophilized cake first needs to be reconstituted with the sterile diluent and then the dose is immediately diluted with saline to a final concentration not more than 0.45 mg/mL of melphalan. The resultant dilute solutions of melphalan must be injected within 1 hour after reconstitution. Dilute solutions should not be stored in a refrigerator as melphalan precipitates in cold conditions.

Evomela® comprises melphalan hydrochloride and betadex sulfobutyl ether sodium. Evomela® is diluted with 0.9% sodium chloride injection to get a final concentration of 0.45 mg/mL and infused over 30 minutes. The Evomela® admixture solution is stable for only 4 hours at room temperature.

Reconstitution and mixing with diluents consume both time and effort. In addition, the reconstitution and mixing must be done in designated aseptic hoods to avoid contamination.

Ready-to-dilute solutions of melphalan would offer a significant advantage in the hospital setting and would minimize the risk of contamination. The process with such ready-to-dilute solutions would be simply to draw the solution into a syringe from a vial and then push the solution inside the syringe into IV bags with isotonic diluents.

A ready-to-dilute solution which is stable for an adequate time would ensure minimum preparation time for injection. Such a ready-to-dilute solution would not need to be diluted in advance, nor would it need to be kept in a refrigerator, as dilution for injection can be done within a minute or so.

WO 2017/085696 A1 discloses a propylene glycol free formulation of melphalan containing cyclodextrin and solvents. However, the melphalan is a lyophilized powder and the diluent comes with cyclodextrin and other excipients, and the reconstitution step is involved before diluting with isotonic fluids for infusion. Furthermore, after reconstitution and further dilution, the formulation started degrading considerably at 6 hours.

U.S. Ser. No. 10/537,520 discloses a liquid parenteral formulation consisting of melphalan hydrochloride, a solvent selected from dimethylacetamide, polyethylene glycol, ethanol, propylene glycol, and glycerine, and antioxidants selected from monothioglycerol, L-cysteine, and ascorbic acid. The non-aqueous formulations were stable for 6 months at 2-8° C. but, at 25° C./60% RH, total impurities increased significantly after 6 months. Furthermore, it is known that the solvents, like dimethylacetamide, leach the chemicals from infusion bags and IV infusion sets and, thus, it is not always advisable to use such solvents.

WO 2019/130228 A1 discloses a non-aqueous, ready to dilute liquid pharmaceutical composition comprising (i) melphalan or a pharmaceutically acceptable salt thereof and (ii) polyoxyethylene sorbitan fatty acid esters. WO 2019/130228 A1 claims the diluted composition is stable up to, at most, about 24 hours. The surfactants made of polyoxyethylene sorbitan fatty acid esters cause severe-hypersensitivity reactions that mandate pretreatment with anti-histamines.

U.S. Ser. No. 10/682,326 discloses a non-aqueous liquid, ready-to-dilute formulation consisting essentially of melphalan; and a solvent selected from dimethylacetamide, polyethylene glycol, ethanol, propylene glycol, dimethyl sulfoxide, N-methylpyrrolidone, and glycerol; wherein said formulation is free of antioxidants, organic acid, and added chloride ions. While U.S. Ser. No. 10/682,326 claims that its formulations are stable for significant periods of time without significant physical instability, it only provides data for 6 months at refrigerated conditions for formulations consisting of melphalan HCl, propylene glycol, polyethylene glycol, and ethanol (dehydrated). Furthermore, such formulations were only stable at room temperature for up to 4 hours after dilution with 0.9% sodium chloride.

U.S. Ser. No. 10/864,183, U.S. Ser. No. 10/940,128, and U.S. Ser. No. 11/020,363 disclose lyophilized compositions of melphalan with a cyclodextrin sulfoalkyl ether derivative, such as sulfobutyl ether cyclodextrin. Such compositions exhibit stability after reconstitution up to 48 hours when stored in refrigerated conditions. However, the reconstituted solutions are not stable for more than 10 hours when maintained at room temperature.

U.S. Pat. Nos. 8,410,077 and 9,200,088 discloses compositions containing sulfoalkyl ether cyclodextrins in their pure form.

U.S. Pat. No. 9,493,582 and U.S. Ser. No. 10/040,872 disclose compositions containing low-chloride alkylated cyclodextrin compositions, including a sulfoalkyl ether cyclodextrin, and processes for preparing thereof.

EP 0317281 B1 describes an injectable formulation of melphalan comprising as two separate components a) lyophilized melphalan HCl and, preferably, a matrix forming agent, such as polyvinylpyrrolidone, and b) a solvent-diluent comprising a citrate, propylene glycol, water, and ethanol. EP 0317281 B1 says nothing about the stability of its reconstituted melphalan product.

There still remains an urgent need for a liquid formulation of melphalan with no harmful solvents, such as dimethylacetamide, that is stable with controlled impurity levels for extended periods.

### SUMMARY OF THE INVENTION

The invention relates to stable, liquid, pharmaceutical compositions comprising, consisting of, or consisting essentially of melphalan, at least one cyclodextrin, at least one non-aqueous solvent, water and/or at least one aqueous buffer, at least one antioxidant, optionally, at least one chelating agent, and, optionally, at least one inorganic salt.

The invention further relates to ready-to-dilute stable, liquid pharmaceutical compositions of the invention.

The invention further relates to methods of treating cancers, including multiple myeloma, advanced ovarian adenocarcinoma, early and advanced breast cancer, childhood neuroblastoma, and polycythaemia vera.

The invention also relates to methods of making the stable, liquid pharmaceutical compositions of the invention.

The invention further relates to dosage forms containing the stable, liquid pharmaceutical compositions of the invention.

### DETAILED DESCRIPTION OF THE INVENTION

The invention relates to stable, liquid pharmaceutical compositions comprising, consisting of, or consisting essentially of melphalan, at least one cyclodextrin, at least one non-aqueous solvent, water and/or at least one aqueous buffer, at least one antioxidant, optionally, at least one chelating agent, and, optionally, at least one salt.

A “stable” composition of the invention means a pharmaceutical composition having sufficient stability at room temperature conditions to have utility as a pharmaceutical product. Preferably, a “stable” composition of the invention has sufficient stability to allow storage at preferably about 2-25° C., more preferably about 2-10° C., most preferably about 2-8° C., for ≥6 months, ≥1 year (e.g., ≥2 years, 3 years, ≥4 years), with ≥90% of un-degraded melphalan (e.g., ≥91%, ≥92%, ≥93%, ≥94%, ≥95%, ≥96%, ≥97%, ≥98%, ≥99%) and with total impurities ≤6% (e.g., ≤5%, ≤4%, ≤3%, ≤2%, ≤1%, ≤0.1%), as determined by HPLC at a wavelength of 260 nm. The “stable” compositions of the invention may have a potency of ≥90% (e.g., ≥85%, ≥90%, ≥98%) of the melphalan when stored at room temperature or refrigerated conditions.

The amount of melphalan present in the compositions of the invention may vary depending on the amount necessary for therapeutic administration. For example, the compositions of the invention may contain about 1-100 mg/mL melphalan, such as, for example, about 2-75 mg/mL, about 5-50 mg/mL, about 10-25 mg/mL, about 15-20 mg/mL. These dosage ranges are not intended to be limiting. A practitioner skilled in the art may likewise administer suitable compositions of the invention in single or divided doses, according to the desired therapeutic effect. Thus, in certain clinical situations it may be desirable to administer compositions of the invention to give initial high levels of the drug, followed by lower maintenance doses. The term “melphalan” includes melphalan or its pharmaceutically acceptable salts or esters (e.g., melphalan HCl).

The compositions of the invention also contain at least one cyclodextrin. Particularly preferred cyclodextrins are hydroxypropyl-β-cyclodextrin, hydroxypropyl-γ-cyclodextrin, or mixtures thereof. Most preferably, the cyclodextrin is hydroxypropyl-β-cyclodextrin. In some embodiments, the compositions of the invention do not contain a charged cyclodextrin and/or a sulfoalkyl ether cyclodextrin (e.g., sulfobutyl ether cyclodextrin) or a derivative thereof, such as those disclosed in U.S. Ser. No. 10/864,183, U.S. Ser. No. 10/940,128, U.S. Ser. No. 11/020,363, U.S. Pat. Nos. 8,410,077, 9,200,088, 9,493,582, and U.S. Ser. No. 10/040,872.

The at least one cyclodextrin may be present in the compositions of the invention in any amount effective to stabilize melphalan. The cyclodextrin is typically present in the composition in an amount up to about 20 wt % (e.g., 1-15, 2-13, 3-11, 4-9, 5-7 wt %). More preferably, the cyclodextrin is present in the composition in an amount ranging from about 1-5 wt %.

The compositions of the invention also include at least one non-aqueous solvent. The term “non-aqueous solvent” means a solvent that contains minimal or no water. The term “minimal” in the context of the non-aqueous solvent means a solvent that contains less than 1.0% v/v, preferably less than 0.1% v/v, more preferably less than 0.01% v/v, even more preferably less than 0.001% v/v, water. Particularly preferred non-aqueous solvents are polyethylene glycols (PEGs) having an average molecular weight ranging from 400-600 g/mol (e.g., PEG 400, PEG 500, PEG 600). Also preferred are compositions which contain PEG 400, PEG 300, or mixtures thereof as the non-aqueous solvent. Propylene glycol is another example of a non-aqueous solvent that may be present in the compositions of the invention. In other embodiments, the compositions of the invention do not contain propylene glycol.

The compositions of the invention may comprise up to about 100% v/v of the at least one non-aqueous solvent, such as up to about 95% v/v, up to about 90% v/v, up to about 75% v/v, up to about 60% v/v, or up to about 45% v/v. Preferably, the compositions of the invention comprises about 5-100% v/v of the at least one non-aqueous solvent, such as about 10-70% v/v, about 20-60% v/v, or about 30-50% v/v. Preferably, the compositions of the invention comprise about 30-95% v/v (e.g., about 40% v/v) of the at least one non-aqueous solvent.

The compositions of the invention also contain water and/or at least one aqueous buffer. The water and/or at least one aqueous buffer may be present in the compositions of the invention in an amount ranging from about 2-20% v/v, preferably about 2-15% v/v, most preferably about 2-10% v/v (e.g., 3-9% v/v, 4-8% v/v, 5-7% v/v). Non-limiting examples of aqueous buffers that may be used in the compositions of the invention include, for example, sodium citrate/citric acid in water, sodium acetate and acetic acid in water, etc. Most preferably, the compositions of the invention contain water in an amount of about 5% v/v.

The compositions of the invention also include at least one antioxidant. For example, the antioxidant may be selected from the group consisting of monothioglycerol (MTG), cysteine (e.g., L-cysteine HCl), tocopherol and its derivatives (e.g., α-tocopherol, D-α-tocopheryl polyethylene glycol 1000 succinate (Vitamin E TPGS or simply TPGS)), butylated hydroxyl anisole (BHA), butylated hydroxyl toluene (BHT), inorganic sulfates (e.g., sodium sulfate and metabisulfite), aromatic compounds (e.g., gallic acid, gentistic acid, vannilic acid), and mixtures thereof. Preferably, a combination of antioxidants may be used in the compositions of the invention, such as BHA-MTG and BHA-TPGS, more preferably BHA-MTG. The antioxidants may be present in the compositions of the invention in an amount ranging from about 0.01-10 wt %, preferably from about 0.02-5 wt %, more preferably about 0.03-1 wt %, and even more preferably about 0.04-0.5 wt %. In some embodiments, the compositions of the invention do not contain monothioglycerol.

The compositions of the invention may also include at least one chelating agent, such as ethylenediaminetetraacetic acid (EDTA) or its salts (e.g., disodium EDTA). The chelating agent may be present in the compositions of the invention in an amount ranging from about 0.01-0.5 mg/mL (e.g., about 0.05-0.4 mg/mL, about 0.1-0.3 mg/mL, about 0.15-0.2 mg/mL). Preferably, the chelating agent is EDTA disodium salt and the EDTA disodium salt is present in an amount ranging from 0.01-0.2 mg/mL, most preferably about 0.1 mg/mL.

The compositions of the invention may also include a stabilizing amount of at least one inorganic salt, such as sodium chloride, magnesium chloride, and other pharmaceutically acceptable salts. Preferably, the inorganic salt is a chloride salt, such as sodium chloride and/or magnesium chloride, most preferably, magnesium chloride. The inorganic salt may be present in the compositions of the invention in an amount ranging from about 0.01-5 wt %, preferably from about 0.1-2.5 wt %, more preferably about 1-2 wt %.

The compositions of the invention may also include at least one pharmaceutically acceptable excipient, such as surfactants, antimicrobials, preservatives, alkalizers and pH modifying agents, and the like. Exemplary surfactants include, but are not limited to, poloxamers, tweens, spans, and other fatty acid esters. Exemplary antimicrobials include, but are not limited to, benzoic acid, methyl and propyl parabens. Exemplary preservatives include, but are not limited to, parabens, benzoates, alcohols, quaternary ammonium salts. Exemplary alkalizers and pH modifying agents include, but are not limited to, sodium hydroxide, potassium hydroxide, tromethamine, lysine, arginine, glycine, meglumine and other appropriate bases. Preferably, the alkalizer is sodium hydroxide and it is present in the compositions of the invention in an amount ranging from about 0.01-5 wt %, preferably from about 0.1-4 wt %, more preferably about 1-3 wt %, and even more preferably about 1.5-2 wt %.

The compositions of the invention may also include additional solubilizers, such as, for example, salt forming agents, complexing agents, polymeric micelle forming agents, and other appropriate excipients which aid solubilization of drugs.

The pharmaceutically acceptable excipient and/or solubilizers may be present in the compositions of the invention in amounts typically known and used in the pharmaceutical formulation art.

Liquid dosage forms according to the present invention may be “ready-to-use” or “ready to dilute” formulations. The term “ready-to-use” composition as used herein refers to a composition of the invention which avoids reconstitution and may require dilution with a suitable diluent before administration to the patient. The term “ready-to-dilute” composition as used herein refers to a composition of the invention that requires a single dilution before administering to a patient.

### Methods of Treatment

The invention also relates to methods of treating cancers, which comprises administering an effective amount of the compositions of the invention to a mammal (e.g., human, equine, bovine, ovine, canine, feline, porcine) in need thereof. For example, compositions of the invention may be used in the treatment of multiple myeloma, advanced ovarian adenocarcinoma, early and advanced breast cancer, childhood neuroblastoma, and polycythaemia vera.

The compositions of the invention can be administered to a mammal in need thereof parenterally, such as by subcutaneous, intramuscular, or intravenous routes. The compositions may be administered directly, without dilution, for example, by injection as short infusion. Alternatively, they may be diluted further with pharmaceutically acceptable diluents (e.g., solutions of dextrose, sodium chloride, sodium lactate, an amino acid, glycerol, sorbitol, dextrose, mannitol, and mixtures thereof) before injection. After dilution with these solutions, the volume for infusion may be 500 ml or less, 75 ml or less, 50 ml or less, with an infusion time of 20 min or less, 15 minutes or less or 10 minutes or less.

The compositions of the invention may be administered either alone or in combination with other therapeutic agents having similar or different biological activities. For example, compositions of the invention may be administered in a combination therapy, i.e., either simultaneously in single or separate dosage forms or in separate dosage forms within seconds, minutes, hours, or days of each other. Examples of therapeutic agents used in such combination therapies include without limitation, chemotherapeutic agents, immunosuppressive agents, immunostimulatory, antipyretic, cytokines, opioids, cytotoxic agents, nucleolytic compounds, radioactive isotopes, receptors, pro-drug activating enzymes, which may be naturally occurring or produced by recombinant methods, anti-inflammatory or anti-rheumatic agents, antibiotics, protease inhibitors, growth factors, osteo-inductive factors, analgesics, anticonvulsants, antidepressants, natural opium alkaloids, anti-epileptics, non-selective monoamine reuptake inhibitors, anilides, diphenylpropylamine derivatives, acetic acid derivatives and related substances, platelet aggregation inhibitors excluding heparin, carboxamide derivatives, propionic acid derivatives, salicylic acid derivatives, local anesthetics, topical non-steroidal anti-inflammatory compounds, opium alkaloids and derivatives, anesthetics for topical use, drugs used in opioid dependence, hydantoin derivatives, oripavine derivatives, phenylpiperidine derivatives, proton pump inhibitors (e.g., omeprazole and/or any of its stereoisomers), and the like.

### Preparation of Compositions of the Invention

The compositions of the invention can be prepared by a variety of techniques known in the art. For example, the method may comprise combining melphalan with the at least one cyclodextrin, at least one non-aqueous solvent, at least one chelating agent, one stabilizing salt and at least one antioxidant. The components of the compositions of the invention may be combined in a single solution or prepared as separate solutions that are then combined.

### Dosage Forms Containing Compositions of the Invention

Compositions of the invention can be provided in unit presentations. Each unit presentation can contain a single dose or multiple-doses of a composition of the invention. For example, a unit containing a composition of the invention may contain one, two, three, four, five, six, seven, eight, nine, ten, or more doses. The units may be provided in any suitable type of sealed container known to those in the art. For example, the units may be packaged and provided in vials, ampoules, syringes, sealed bottles, or sealed bags made of pharmaceutically acceptable material, such as glass or pharmaceutically acceptable plastic.

The sealed units containing the compositions of the invention may be stable for storage for extended periods prior to administration. For example, the sealed units containing the compositions of the invention may be stable at refrigerated conditions (about 2-8° C.) for extended periods of time (e.g., ≥1 year, ≥>2 years, ≥>3 years, ≥>4 years), with minimal degradation of the melphalan (e.g., it retains ≥90%, ≥91%, ≥92%, ≥93%, ≥94%, ≥95%, ≥96%, ≥97%, ≥98%, ≥99% of un-degraded melphalan) and with impurities less than or equal to acceptable limits (e.g., total impurities ≤6%, ≤5%, ≤4%, ≤3%, ≤2%, ≤1%, ≤0.1%), as determined by HPLC.

The units containing the compositions of the invention may be administered to a mammal in need thereof of melphalan treatment parenterally, such as by subcutaneous, intramuscular, or intravenous routes.

### EXEMPLARY EMBODIMENTS OF THE INVENTION

E1. A stable, liquid pharmaceutical composition comprising, consisting essentially of, or consisting of:
a) melphalan;
b) at least one cyclodextrin;
c) at least one non-aqueous solvent;
d) water and/or at least one aqueous buffer;
e) at least one antioxidant;
f) optionally, at least one chelating agent; and
g) optionally, at least one inorganic salt.

E2. The composition of E1, wherein the melphalan is present in the composition in an amount ranging from 1-100 mg/mL.

E3. The composition of E1, wherein the cyclodextrin is selected from the group consisting of hydroxy propyl-β-cyclodextrin, hydroxy propyl-γ-cyclodextrin, and mixtures thereof.

E4. The composition of any one of E1-E3, wherein the cyclodextrin is hydroxyl propyl-β-cyclodextrin.

E5. The composition of E1 or E2, wherein the composition does not contain a charged cyclodextrin and/or a sulfoalkyl ether cyclodextrin or derivative thereof.

E6. The composition of any one of E1-E5, wherein the at least one cyclodextrin is present in the composition in an amount ranging from about 1-20% w/v.

E7. The composition of any one of E1-E6, wherein the non-aqueous solvent is selected from the group consisting of PEG 300, 400, PEG 600, and mixtures thereof.

E8. The composition of E7, wherein the non-aqueous solvent is PEG 400.

E9. The composition of E7, wherein the non-aqueous solvent is PEG 300.

E10. The composition of any one of E1-E9, wherein the water and/or at least one aqueous buffer is present in the composition in an amount of up to about 40% v/v.

E11. The composition of E10, wherein the water and/or at least one aqueous buffer is present in the composition in an amount of about 2-20% v/v.

E12. The composition of any one of E1-E9, wherein the water is present in the composition in an amount of about 5% v/v.

E13. The composition of E12, wherein the composition does not contain any aqueous buffer other than water.

E14. The composition of any one of E1-E13, wherein the antioxidant is selected from the group consisting of monothioglycerol, cysteine, tocopherol or derivative thereof, butylated hydroxyl anisole, butylated hydroxyl toluene, inorganic sulfates, aromatic compounds, and mixtures thereof.

E15. The composition of any one of E1-E14, wherein the antioxidant is a combination of butylated hydroxyl anisole-monothioglycerol or butylated hydroxyl anisole-D-α-tocopherol polyethylene glycol 1000 succinate, preferably butylated hydroxyl anisole-monothioglycerol.

E16. The composition of any one of E1-E15, wherein the antioxidant is present in the composition in an amount ranging from about 0.04-0.5% w/v.

E17. The composition of any one of E1-E16, wherein the chelating agent is EDTA or its salts.

E18. The composition of E17, wherein the EDTA is disodium EDTA.

E19. The composition of any one of E1-E18, wherein the chelating agent is present in the composition in an amount ranging from about 0.01-0.5 mg/mL, preferably 0.01-0.2 mg/mL, most preferably about 0.1 mg/mL.

E20. The composition of any one of E1-E19, wherein the inorganic salt is a chloride salt.

E21. The composition of E20, wherein the chloride salt is sodium chloride and/or magnesium chloride, preferably, magnesium chloride.

E22. The composition of any one of E1-E21, wherein the inorganic salt is present in the composition in an amount ranging from about 0.01-5 wt %.

E23. The composition of any one of E1-E22, further comprising at least one pharmaceutically acceptable excipient.

E24. The composition of E23, wherein the pharmaceutically acceptable excipient is selected from the group consisting of at least one surfactant, at least one antimicrobial, at least one preservative, at least one alkalizer and pH modifying agent, and mixtures thereof.

E25. The composition of E24, wherein the alkalizer is sodium hydroxide.

E26. The composition of E24 or E25, wherein the alkalizer is present in the composition in an amount of about 0.01-2% v/v.

E27. The composition of E1, wherein:
the melphalan is present in the composition in an amount ranging from 1-100 mg/mL;
the at least one cyclodextrin is selected from the group consisting of hydroxy propyl-β-cyclodextrin, hydroxy propyl-γ-cyclodextrin, and mixtures thereof;
the at least one non-aqueous solvent is selected from the group consisting of PEG 300, 400, PEG 600, and mixtures thereof;
water is present;
the at least one antioxidant is selected from the group consisting of monothioglycerol (MTG), cysteine, tocopherol or derivative thereof, butylated hydroxyl anisole (BHA), butylated hydroxyl toluene (BHT), inorganic sulfates, aromatic compounds, and mixtures thereof;
the at least one chelating agent is EDTA or its salts; and
the at least one inorganic salt is a chloride salt,
   wherein the composition further comprises an alkalizer selected from sodium hydroxide.

E28. The composition of E27, wherein:
the at least one cyclodextrin is hydroxy propyl-β-cyclodextrin;
the at least one non-aqueous solvent is PEG-300;
the at least one antioxidant is a combination of butylated hydroxyl anisole-monothioglycerol (BHA-MTG) or butylated hydroxyl anisole-D-α-tocopherol polyethylene glycol 1000 succinate (BHA-TPGS);
the at least one chelating agent is disodium EDTA; and
the at least one inorganic salt is sodium chloride and/or magnesium chloride.

E29. The composition of E28, wherein:
the at least one antioxidant is a combination of BHA-MTG; and
the at least one inorganic salt is magnesium chloride.

E30. The composition of E27, wherein:
the at least one cyclodextrin is hydroxy propyl-β-cyclodextrin;
the at least one non-aqueous solvent is PEG-400;
the at least one antioxidant is a combination of butylated hydroxyl anisole-monothioglycerol (BHA-MTG) or butylated hydroxyl anisole-D-α-tocopherol polyethylene glycol 1000 succinate (BHA-TPGS);
the at least one chelating agent is disodium EDTA; and
the at least one inorganic salt is sodium chloride and/or magnesium chloride.

E31. The composition of E30, wherein:
the at least one antioxidant is a combination of BHA-MTG; and
the at least one inorganic salt is magnesium chloride.

E32. The composition of any one of E27-E31, wherein the composition comprises: about 1-5 wt % of the at least one cyclodextrin;
about 30-95% v/v of the at least one non-aqueous solvent;
about 5% v/v of the water;
about 0.04-0.5 wt % of the at least one antioxidant;
about 0.1 mg/mL of the at least one chelating agent;
about 1-2 wt % of the at least one inorganic salt; and
about 1.5-2 wt % of the alkalizer.

E33. The composition of any one of E27-E32, wherein the composition does not contain a charged cyclodextrin and/or a sulfoalkyl ether cyclodextrin or derivative thereof.

E34. The composition of any one of E1-E33, wherein the composition contains total impurities 56% in the composition resulting from the degradation of melphalan in the composition, as determined by HPLC at a wavelength of 260 nm, at about 2-8° C. for ≥1 years.

E35. A sealed unit dose of the composition of any one of E1-E34.

E36. A method of treating cancer comprising, consisting essentially of, or consisting of the administration of an effective amount of a composition of anyone of E1-E35 to a mammal in need thereof.

E37. The method of E36, wherein the cancer is selected from the group multiple myeloma, advanced ovarian adenocarcinoma, early and advanced breast cancer, childhood neuroblastoma, and polycythaemia vera.

E38. The method of E36 or E37, wherein the composition is not diluted before administering to the mammal.

E39. The method of E36 or E37, wherein the composition is diluted with at least one pharmaceutically acceptable diluent before administration.

E40. The method of any one of E36-E39, wherein the composition is administered parenterally.

E41. The method of E40, wherein the parenteral administration is by a subcutaneous, intramuscular, or intravenous route.

### EXPERIMENTAL

### Materials and Characterization

Melphalan was obtained from Apothecon Pharmaceuticals Pvt. Ltd, India. PEG 400 (USP/NF) was obtained from Merck. PEG 300 and propylene glycol (PG) were obtained from Sigma Aldrich (analytical or meeting USP specifications). Tocopherol, Vitamin E TPGS, and monothioglycerol, Cysteine Hydrochloride, Magnesium chloride were of analytical grade and were purchased from Sigma-Aldrich. Hydroxy propyl-β-cyclodextrin (HPβCD) (Kleptose HPB, parenteral grade) was obtained from Roquette, Germany.

Stability of melphalan formulations was assessed using HPLC method, which used gradient elution as mentioned below in Table 1:

### Chromatographic Conditions:

### Related Substances and Assay:

Column: 10×4.6 mm, 5 μm (Avantor ACE)
Flow Rate: 1.5 mL/min
Runtime: 35 min
Injection Volume: 20 μl
Auto Sampler: 25° C.
Needle Wash: Methanol
Column Oven Temperature: 25° C.
Wavelength: 260 nm

### Mobile Phase A:

A mixture of 5 volumes of acetonitrile for chromatography and 95 volumes of water containing 0.01% v/v of triethylamine, 0.05% m/m of ammonium acetate, and 0.05% v/v of glacial acetic acid.

### Mobile Phase B:

A mixture of 40 volumes of water containing 0.01% v/v of triethylamine, 0.05% m/m of ammonium acetate and 0.05% v/v of glacial acetic acid, and 60 volumes of acetonitrile.

### Diluent: Methanol

### Gradient:

TABLE 1
Gradient Program for HPLC method

| Time | Flow rate | | |
|---|---|---|---|
| (Minutes) | (mL/Minute) | Mobile Phase A (%) | Mobile Phase B (%) |
| 0 | 1.5 | 100 | 0 |
| 20 | 1.5 | 0 | 100 |
| 25 | 1.5 | 0 | 100 |
| 27 | 1.5 | 100 | 0 |
| 35 | 1.5 | 100 | 0 |

### Standard and Sample Solutions Preparation:

### Related Substances (Impurities)

Melphalan HCl Standard Solution (1 mg/ml):

Weighed and transferred 20 mg of melphalan in a 20 mL volumetric flask dissolved and diluted to volume with the Methanol (1 mg/mL).

Melphalan HC Standard Solution (0.001 mg/mL):

Transfer 0.1 mL of melphalan HCl standard solution (1 mg/mL) into 100 mL volumetric flask and dilute to volume with diluent and mix.

Melphalan HCl Standard Solution (0.001 mg/mL):
Transfer 0.5 mL of melphalan HCl standard solution (1 mg/mL) into 100 mL volumetric flask and dilute to volume with diluent and mix.

### For Assay:

Melphalan HCl Standard Solution (0.1 mg/mL):

Weigh and transfer 10 mg of melphalan HCl into a 100 mL volumetric flask, dissolve the material and diluted to volume with the Methanol (0.1 mg/mL).

### Sample Preparation Details:

### For Related Substance:

Dilute melphalan injection with methanol to get to solution with 1 mg/mL of melphalan.

### For Related Assay:

Dilute melphalan injection with methanol to get to solution with 0.1 mg/mL of melphalan.

### Example 1: Melphalan Formulations with Different Non-Aqueous Solvents

Formulations T-01 to T-03 were prepared by mixing all excipients and solvents first and sonicating them to get a clear solution. Melphalan was added to the solvent-excipients mixture and sonicated to get a clear solution. Table 2 shows the composition of Formulations T-01 to T-03 and their stability. Additionally, Table 2 shows the stability of these formulations, as determined by HPLC, initially and at 8 days and 4 months, 13 days at 40° C./75% RH, and at 2 months, 22 days and 6 months at 25° C./60% RH.

TABLE 2
Compositions for Formulations T-01 to T-03 and Their Stability

| Ingredients | T-01 | | T-02 | | T-03 | |
|---|---|---|---|---|---|---|
| Melphalan HCl | 10 | mg | 10 | mg | 10 | mg |
| Hydroxy Propyl-β- | 100 | mg | 100 | mg | 100 | mg |
| Cyclodextrin (HPβCD) | | | | | | |
| MTG | 5 | mg | 5 | mg | 5 | mg |

| | | | | |
|---|---|---|---|---|
| Propylene Glycol | 1 | mL | — | — |

| | | | | |
|---|---|---|---|---|
| PEG 400 | — | 1 | mL | — |

| | | | | |
|---|---|---|---|---|
| PEG-300 | — | — | 1 | mL |

Assay (%)

| | | | |
|---|---|---|---|
| Initial | 97.05 | 97.70 | 92.60 |
| 8 days 40° C./75% RH | 92.84 | 52.94 | 79.42 |
| 4 months 13 days | 47.02 | 37.45 | 74.07 |
| 40° C./75% RH | | | |
| 2 months 22 days | 96.66 | 71.27 | 86.06 |
| 25° C./60% RH | | | |
| 6 months 25° C./60% RH | 87.97 | 64.27 | 81.57 |

After six months at 25° C./60% RH, potency decreases to below 90% of initial assay for all formulations. However, those with propylene glycol and PEG-300 were marginally better.

### Example 2: Formulations with PEG-300 or Propylene Glycol, with and without Water

For Formulations F-10 and F-11 (Table 3), MTG, TPGS, and hydroxy propyl-β-cyclodextrin (HPβCD) were first dissolved in PEG-300 followed by addition of melphalan. The mixtures were sonicated to get clear solutions. For Formulation F-12 (Table 3), sodium chloride and HP CD were first dissolved in water and then mixed with PEG-300 solution with MTG and TPGS. Finally, melphalan was added to the mixture and sonicated to get a clear solution. The stability of Formulations F-10, F-11, and F-12 was determined by HPLC, at 3 months (25° C./60% RH) (Table 4). Surprisingly, the inclusion of water decreases impurities significantly, 30-40%, and the number of impurities >0.5% reduces to 1 after addition of water compared to 3 for the formulations without water.

TABLE 3
Compositions for Formulations F-10 to F-12

| | | | |
|---|---|---|---|
| Ingredients | F-10 | F-11 | F-12 |

| | | | | | | |
|---|---|---|---|---|---|---|
| Melphalan HCl | 10 | mg | 10 | mg | 10 | mg |
| HPβCD | 100 | mg | 100 | mg | 50 | mg |
| MTG | 5 | mg | 5 | mg | 5 | mg |

| | | | | |
|---|---|---|---|---|
| Vit. E TPGS | 1.25 | 2.5 | 2.5 | mg |

| | | | | | | |
|---|---|---|---|---|---|---|
| PEG-300 | 1 | mL | 1 | mL | 0.95 | mL |

| | | | | |
|---|---|---|---|---|
| Sodium chloride | — | — | 4.5 | mg |
| MilliQ Water | — | — | 0.05 | mL |

TABLE 4
Stability data of Formulations F-10 to F-12

| | Formulation | | |
|---|---|---|---|
| | F-10 | F-11 | F-12 |
| | 3 months | 3 months | 3 months |
| | (25° C./60% RH) | (25° C./60% RH) | (25° C./60% RH) |
| Assay (%) | 91.5 | 91.65 | 90.36 |

Impurities, RRT (%)

| | | | |
|---|---|---|---|
| 0.242 | 1.66 | 1.5 | 1.54 |
| 0.409 | 0.09 | 0.24 | 0.29 |
| 0.476 | 0.05 | 0.1 | 0.12 |
| 0.573 | 0.04 | 0.06 | 0.04 |
| 0.589 | 0.02 | — | 0.05 |
| 0.729 | 0.03 | 0.04 | 0.02 |
| 0.752 | — | — | 0.02 |
| 0.775 | — | 0.02 | — |
| 0.794 | 0.02 | 0.03 | 0.04 |
| 0.824 | 0.02 | 0.03 | 0.04 |
| 0.851 | 0.02 | 0.02 | 0.02 |
| 0.947 | — | — | 0.14 |
| 0.96 | 0.09 | 0.07 | |
| 1.068 | 0.24 | 0.16 | — |
| 1.112 | 0.04 | — | 0.1 |
| 1.117 | — | — | 0.39 |
| 1.129 | 0.12 | 0.41 | — |
| 1.172 | 0.05 | — | — |
| 1.203 | 0.54 | 0.35 | 0.32 |
| 1.261 | 0.03 | — | — |
| 1.290 | 1.36 | 1.4 | 0.24 |
| 1.313 | — | — | 0.1 |
| 1.365 | 0.04 | 0.08 | 0.02 |
| 1.404 | 0.14 | 0.2 | 0.08 |
| 1.439 | — | — | 0.02 |
| 0.146 | 0.03 | 0.02 | |
| 1.530 | 0.04 | 0.03 | — |
| 1.571 | 0.05 | 0.03 | — |
| 1.623 | 0.23 | 0.26 | 0.15 |
| 1.633 | 0.02 | 0.02 | — |
| 1.664 | 0.04 | 0.04 | 0.02 |
| 1.692 | 0.04 | — | — |
| 1.726 | 0.08 | 0.04 | 0.03 |
| 1.754 | — | — | 0.03 |
| 1.785 | 0.09 | 0.05 | — |
| 1.857 | 0.02 | 0.03 | — |
| 1.944 | 0.02 | 0.02 | — |
| Total | 5.26 | 5.25 | 3.82 |
| Impurities (%) | | | |

For Formulation F-15 (Table 5), sodium chloride and HPβCD were first dissolved in water and MTG-TPGS in propylene glycol (PG). The water and PG solutions were mixed, followed by addition of melphalan HCl. The mixture was sonicated to get a clear solution. Table 6 demonstrates that the inclusion of water in the PG-based formulation did not improve stability as it was observed with Formulation F-12 containing PEG-300. This indicates that the combination of solvent and water is preferred for stability, and not just merely inclusion of water in the formulation.

TABLE 5
Formulation F-15 with Propylene Glycol and Water

| Ingredient | Qty mg/mL |
|---|---|
| Melphalan HCl | 10 |
| HPβCD | 50 |
| MTG | 5 |
| Vit. E TPGS | 2.5 |
| Sodium Chloride | 4.5 |
| MilliQ water | 0.05 mL |
| Propylene Glycol | 0.95 mL |

TABLE 6
Stability data of Formulation F-15

| Parameters | F-15 |
|---|---|
| Assay (%) | 87.74 |

Impurities, RRT (%)

| | |
|---|---|
| 0.55 | 2.34 |
| 0.74 | 8.13 |
| 1.22 | 1.33 |
| Total impurities (%) | 13.44 |

### Example 3A: Formulations with EDTA

The same process used in Example 2 to make Formulation F-12 was used to make Formulation F-51 (Table 7). The stability of Formulation F-51 was determined by HPLC, initially (To) and 25° C./60% RH at 3 and 6 months, and 15° C. at 6 months (Table 8), and 40° C./75% RH at 1 month (Table 9).

TABLE 7
Composition for Formulation F-51

| | F-51 | |
|---|---|---|
| Ingredients | Qty/mL | |
| Melphalan HCl | 10 | mg |
| HPβCD | 50 | mg |
| EDTA, Calcium Sodium | 0.1 | mg |
| Vit. E TPGS | 2.5 | mg |
| MTG | 5 | mg |
| Sodium Chloride | 4.5 | mg |
| MilliQ water | 0.05 | mL |
| PEG-300 | 0.95 | mL |

TABLE 8
Stability data of Formulation F-51

| | F-51 | | | |
|---|---|---|---|---|
| Test | | 3 months, | 6 months, | 6 months, |
| parameters | T₀ | 25° C./60% RH | 25° C./60% RH | 15° C. |
| Assay (%) | 100.48 | 97.87 | 95.54 | 99.48 |

Impurities, RRT (%)

| | | | | |
|---|---|---|---|---|
| 0.216 | 0.01 | — | — | — |
| 0.242 | — | 0.06 | 0.08 | 0.08 |
| 0.302 | — | 0.25 | 0.25 | 0.13 |
| 0.533 | — | 0.10 | 0.19 | 0.06 |
| 1.054 | 0.28 | 0.04 | — | — |
| 1.097 | — | — | — | — |
| 1.129 | — | 0.15 | 0.21 | 0.1 |
| 1.204 | — | 0.77 | 0.70 | 0.05 |
| 1.243 | — | 0.26 | 0.22 | — |
| 1.252 | — | 0.05 | — | 0.1 |
| 1.544 | — | — | 0.13 | — |
| 2.247 | — | 0.02 | 0.02 | — |
| Total | 0.41 | 2.03 | 2.22 | 0.65 |
| Impurities | | | | |
| (%) | | | | |

TABLE 9
Formulation F-51 stability data higher temperature
F-51 stability at higher temperature

| RRT | Initial | 1 month 40° C./75% RH |
|---|---|---|
| Assay (%) | 100.48 | 90.43 |

Impurities, RRT (%)

| | | |
|---|---|---|
| 0.216 | 0.01 | — |
| 0.242 | — | 0.23 |
| 0.272 | — | — |
| 0.302 | — | 0.47 |
| 0.418 | 0.01 | 0.01 |
| 0.446 | — | 0.01 |
| 0.514 | 0.02 | — |
| 0.535 | — | 0.22 |
| 0.585 | — | — |
| 0.65 | 0.02 | 0.01 |
| 0.753 | — | 0.02 |
| 0.781 | 0.01 | 0.06 |
| 0.826 | — | 0.08 |
| 0.842 | — | 0.09 |
| 0.874 | — | 0.08 |
| 0.898 | 0.01 | 0.06 |
| 0.914 | — | 0.03 |
| 0.931 | — | 0.01 |
| 1.054 | 0.28 | 0.11 |
| 1.129 | — | 0.25 |
| 1.172 | — | 0.01 |
| 1.21 | — | 1.59 |
| 1.253 | — | 0.63 |
| 1.276 | — | 0.03 |
| 1.285 | — | 0.03 |
| 1.362 | — | 0.01 |
| 1.407 | — | 0.06 |
| 1.464 | — | 0.02 |
| 1.514 | — | 0.28 |
| 1.567 | — | 0.01 |
| 1.614 | — | 0.06 |
| 1.677 | — | 0.04 |
| 1.701 | — | 0.03 |
| 1.751 | — | 0.03 |
| 1.855 | — | 0.02 |
| 1.936 | — | 0.06 |
| Total Impurities | 0.41 | 4.67 |
| (%) | | |

Unexpectedly, the inclusion of EDTA resulted in significant improvement in stability. Formulation F-51 with EDTA had about 2.0% impurities after 3 months at 25′C/60% RH compared to 3.85% for Formulation F-12, which did not contain EDTA. Formulation F-51 was found to be stable even after 6 months while retaining assay and impurity profile comparable to 3 months. There was no further degradation after 3 months. Formulation F-51 was not as stable at 40° C./75% RH. Based on this, and the enhanced stability at 25° C./60% RH, this composition of the invention may be well-suited for refrigerated conditions. However, this formulation turned hazy after storing in refrigerated conditions, and needed about 60 minutes to attain clarity.

### Example 3B: Formulations with EDTA and Magnesium Chloride

For Formulations 40013-137 and 40013-138 (Table 10), magnesium chloride (MgCl₂) and HPβCD were first dissolved in water and then mixed with PEG-300 solution with MTG and TPGS. Finally, melphalan was added to the mixture and sonicated to get a clear solution. The stability of Formulations 40013-137 and 40013-138 was determined by HPLC, at 3 months, 40° C./75% RH (Table 11).

TABLE 10
Composition for Formulations 40013-137 and
40013-138 with Magnesium Chloride and EDTA

| | 40013-137 | 40013-138 |
|---|---|---|
| Ingredients | Qty mg/mL | Qty mg/mL |
| Melphalan HCl | 10 | 10 |
| HPβCD | 50 | 50 |
| MTG | 5 | 5 |
| Vit E. TPGS | 2.5 | 2.5 |
| MgCl₂ | 10 | 10 |
| EDTA Ca Na2 | 0.1 | 0.1 |
| MilliQ water | 0.05 mL | 0.08 mL |
| PEG-300 | 0.95 mL | 0.92 mL |

TABLE 11
Stability data of Formulations 40013-137 and 40013-138

3 months, 40° C./75% RH

| Parameters | 40013-137 | 40013-138 |
|---|---|---|
| Assay | 95.10% | 92.50% |

Organic impurities, RRT (%)

| | | |
|---|---|---|
| 0.17 | 0.44 | 0.49 |
| 0.43 | 0.37 | 0.82 |
| 0.80 | 0.09 | 0.14 |
| 0.84 | 0.15 | 0.22 |
| 0.87 | 0.16 | 0.24 |
| 0.91 | 0.16 | 0.23 |
| 0.93 | 0.11 | 0.16 |
| 0.96 | 0.06 | 0.09 |
| 1.10 | 0.08 | 0.13 |
| 1.25 | 0.2 | 0.24 |
| 1.45 | 0.11 | 0.23 |
| 1.47 | 0.16 | 0.11 |
| 1.51 | 0.39 | 0.42 |
| 1.53 | 0.31 | 0.26 |
| 1.55 | 0.22 | 0.23 |
| 1.58 | 0.09 | 0.14 |
| 1.59 | 0.04 | 0.06 |
| Total Impurities % (≥0.10%) | 2.78 | 4.06 |
| Total Impurities (%) | 3.14 | 4.25 |

The results for Formulations 40013-137 and 40013-138 in Table 11 indicate magnesium chloride has better stabilizing effects than NaCl. Tables 10 and 11 also demonstrate that the concentration of water is important, specifically higher water (8% v/v) resulted in increased degradation. Water at 5% or less increases the stabilization of melphalan.

### Example 4: Formulations with Cysteine

Formulations 40013-078A and 40013-078B (Table 12) were made by dissolving L-cysteine, NaOH, NaCl, and cyclodextrin in water and dissolving TPGS in PEG-400. The water phase and PEG 400 were mixed. Finally, melphalan was added to the mixtures and sonicated until a clear solution was formed. For Formulations 40013-83 and 40013-91 (Table 12) the previously described procedure (same as 40013-137-138) was used, with the exception to the addition of TPGS. For Formulations 40013-83 and 40013-91 no TPGS was added, and for Formulation 40013-91 butylated hydroxy anisole (BHA) was dissolved in the PEG-400 instead of TPGS. The stability of Formulations 40013-078A and 40013-078B was determined by HPLC, initially, 70° C. at 5 hours, and 40° C./75% RH at 1 month (Tables 13 and 14).

TABLE 12
Composition for Formulations 40013-078A and 40013-078B

| | 40013-078A | 40013-078B | 40013-083 | 40013-091 |
|---|---|---|---|---|
| | Qty | Qty | Qty | Qty |
| Ingredients | (mg/mL) | (mg/mL) | (mg/mL) | (mg/mL) |
| Melphalan HCl | 10 | 10 | 10 | 10 |
| HPβCD | 50 | 50 | 50 | 50 |
| L-Cysteine HCl | 5 | 10 | 5 | 5 |
| Vit. E TPGS | 2.5 | 2.5 | 0.0 | 0.0 |
| Butylated Hydroxy | 0.0 | 0.0 | 0.0 | 0.6 |
| Anisole (BHA) | | | | |
| Disodium EDTA | 0.1 | 0.1 | 0.1 | 0.1 |
| Sodium Hydroxide | 0.2 | 0.2 | 0.225 | 0.225 |
| Sodium Chloride | 4.5 | 4.5 | 4.5 | 4.5 |
| MilliQ water (mL) | 0.08 | 0.08 | 0.10 | 0.10 |
| PEG-400 (mL) | 0.92 | 0.92 | 0.90 | 0.90 |

TABLE 13
Stability data of Formulation 40013-78A
40013-078A

| | | | 1 month |
|---|---|---|---|
| RRT | Initial | 5 hr 70° C. | 40° C./75% RH |
| Assay (%) | 102.15 | 101.86 | 100.75 |

Impurities, RRT (%)

| | | | |
|---|---|---|---|
| 0.389 | — | 0.26 | — |
| 0.443 | — | — | 0.15 |
| 0.472 | 0.02 | 0.09 | — |
| 0.529 | — | — | 0.22 |
| 0.618 | 0.01 | 0.02 | — |
| 0.655 | — | — | 0.02 |
| 0.771 | 0.02 | 0.02 | — |
| 0.795 | — | — | 0.02 |
| 0.842 | — | — | 0.02 |
| 0.865 | — | — | 0.04 |
| 0.887 | — | — | 0.03 |
| 0.905 | — | — | 0.03 |
| 0.922 | — | — | 0.03 |
| 0.938 | — | — | 0.01 |
| 1.137 | — | — | 0.06 |
| 1.15 | — | 0.02 | 0.02 |
| 1.249 | — | — | 0.11 |
| 1.259 | — | — | 0.01 |
| 1.278 | — | 0.02 | 0.04 |
| 1.293 | 0.07 | 0.05 | — |
| 1.327 | 0.02 | 0.03 | — |
| 1.405 | 0.02 | 0.02 | — |
| Total Impurities (%) | 0.16 | 0.53 | 0.81 |
| Assay (%) | 102.15 | 101.86 | 100.75 |

TABLE 14
Stability data of Formulation 40013-078B
40013-078B

| | | | 1 month |
|---|---|---|---|
| RRT | Initial | 5 hr 70° C. | 40° C./75% RH |
| Assay (%) | 101.51 | 100.50 | 99.96 |

Impurities, RRT (%)

| | | | |
|---|---|---|---|
| 0.389 | 0.02 | 0.4 | — |
| 0.443 | — | — | 0.11 |
| 0.472 | 0.02 | 0.08 | — |
| 0.529 | — | — | 0.23 |
| 0.618 | 0.02 | 0.02 | — |
| 0.655 | — | — | 0.02 |
| 0.771 | — | 0.02 | — |
| 0.795 | — | — | 0.03 |
| 0.842 | — | — | 0.02 |
| 0.865 | — | — | 0.04 |
| 0.887 | — | — | 0.03 |
| 0.905 | — | — | 0.03 |
| 0.922 | — | — | 0.03 |
| 0.938 | — | — | 0.02 |
| 1.137 | — | — | 0.07 |
| 1.15 | — | 0.02 | 0.04 |
| 1.249 | — | — | 0.22 |
| 1.259 | — | — | 0 |
| 1.278 | — | — | 0.03 |
| 1.293 | 0.06 | 0.04 | — |
| 1.327 | 0.02 | — | — |
| 1.405 | 0.02 | — | — |
| Total Impurities (%) | 0.16 | 0.58 | 0.92 |
| Assay (%) | 101.51 | 100.50 | 99.96 |

The inclusion of cysteine HCl in the compositions of the invention had positive effects on stability. Formulations 40013-078A and 40013-078B had excellent stability at 40° C./75% RH after 1 month, and have about 0.8-0.9% impurities compared to >4% for Formulation F-51, which did not contain cysteine.

However, Formulations 40013-078A and 40013-078B failed to maintain physical stability after a month. The solutions showed precipitation at 2 months at 40° C./75% RH and even at 25° C./60% RH after 2.5 to 3.0 months. Similarly, Formulations 40013-083 and 40013-091 also precipitated within 1 month at 40° C./75% RH and stability testing was not performed even at one month time point.

Formulation 40013-099 (Table 15) was made by dissolving L-cysteine, NaOH, NaCl, and cyclodextrin in water and dissolving TPGS in PEG-400. The water phase and PEG 400 were mixed. Finally, melphalan was added to the mixtures and sonicated until a clear solution was formed.

Surprisingly, Formulation 40013-099, which is similar to Formulation 40013-078B, was found to be stable even after 6 months at all three conditions, 6 months at 40° C./75% RH, 9 months at 30° C./65% RH, and 9 months at 25° C./60% RH (Table 16). This could be due to source of PEG 400 used in the formulation and pH of the PEG 400 (Table 17). However, the initial pH of the final formulation did not change between Formulations 40013-078B and 40013-099, values were 2.26 and 2.29. In addition to pH of PEG 400, other factors, like polymer molecular weight and peroxide content, may play a role, and variability of these parameters among the PEG's of different make contribute to varied physical stability of the formulations. The effect of pH was even confirmed when the higher amount of NaOH was added to the formulations similar to Formulations 40013-078B and 40013-099 to make pH to 3.0, but the formulations showed precipitation. Cysteine precipitated when pH was raised from 2.2 to 3.0 and this happened even before the addition of melphalan.

TABLE 15
Composition for Formulation 40013-099
400013-099

| | | |
|---|---|---|
| Ingredients | Qty/mL | Qty/Batch |

| | | | | |
|---|---|---|---|---|
| Melphalan HCl | 10 | mg | 1000 | mg |
| HPβCD | 50 | mg | 5000 | mg |
| EDTA Na2 | 0.1 | mg | 10 | mg |
| L-Cysteine HCl | 10 | mg | 1000 | mg |
| Anhydrous | | | | |
| NaCl | 4.5 | mg | 450 | mg |
| NaOH | 0.225 | mg | 22.5 | mg |
| Vitamin E TPGS | 2.5 | mg | 250 | mg |
| Distilled water | 0.1 | mL | 10 | mL |
| PEG-400 | 0.9 | mL | 90 | mL |

TABLE 16
Stability data of Formulation 40013-099

| | 40013-099 | | | |
|---|---|---|---|---|
| | | 6 months | 9 months | 9 months |
| Parameters | Initial | 40° C./75% RH | 30° C./65% RH | 25° C./60% RH |
| 0.21 | — | 0.16 | — | — |
| 0.29 | — | 0.39 | 0.21 | 0.15 |
| 0.41 | — | 1.4 | 0.63 | 0.34 |
| 0.53 | 0.03 | 0.12 | | — |
| 0.87 | — | 0.13 | 0.06 | 0.03 |
| 0.91 | — | 0.22 | 0.09 | 0.05 |
| 0.93 | — | 0.27 | 0.12 | 0.06 |
| 0.96 | — | 0.22 | 0.1 | 0.05 |
| 1.10 | — | 0.22 | | — |
| 1.13 | — | — | 0.26 | 0.22 |
| 1.25 | 0.05 | 0.13 | 0.08 | 0.05 |
| 1.51 | — | 0.1 | 0.08 | 0.12 |
| 1.52 | — | 0.26 | 0.17 | — |
| 1.53 | — | 0.4 | 0.27 | 0.16 |
| 1.55 | — | — | 0.37 | — |
| 1.56 | — | 0.56 | | 0.21 |
| 1.57 | — | — | 0.43 | 0.24 |
| 1.59 | — | 0.71 | 0.41 | 0.23 |
| 1.60 | — | 0.71 | 0.31 | 0.18 |
| 1.61 | — | 0.61 | 0.19 | 0.11 |
| 1.63 | — | 0.38 | 0.1 | 0.06 |
| 1.64 | — | 0.19 | 0.03 | 0.02 |
| Total Imp | 0.15 | 7.56 | 4.27 | 2.51 |
| Total Imp (≥0.10%) | — | 7.18 | 3.57 | 1.96 |
| Assay | 101.58% | 91.00% | 91.40% | 96.60% |
| pH | 2.297 | 2.57 | | 2.647 |

TABLE 17
pH Data of PEG 400 from Different Makes and Grades

| PEG 400 Make and Grade | pH |
|---|---|
| Spectrum, USP grade, | 4.4 |
| Lot: 2JI0307 | |
| Merck, Emprove, EP | 3.75 |
| Lot: K48004603 | |

### Example 5: Melphalan Formulations Containing BHA as Antioxidant without Cysteine

Formulations 40013-118 and 40013-144 were prepared (Table 18) by first dissolving NaOH, MagCl₂, ETDA, HPβCD in water and BHA, MTG were dissolved in PEG-400. Both water solution and PEG-400 solutions were mixed and finally melphalan HCl was added to the mixture and sonicated until a clear solution was formed.

TABLE 18
Composition for Formulations 40013-
118 and 40013-144 with BHA and MTG

| | | |
|---|---|---|
| Ingredients | 40013-118 Qty/mL | 40013-144 Qty/mL |

| | | | | |
|---|---|---|---|---|
| Melphalan HCl | 10 | mg | 10 | mg |
| HPβCD | 50 | mg | 50 | mg |
| NaOH | 0.4 | mg | 0.4 | mg |
| EDTA Na | 0.1 | mg | 0.1 | mg |
| BHA | 0.2 | mg | 1.0 | mg |
| MTG | 2.5 | mg | 2.5 | mg |
| MgCl₂ | 10 | mg | 10 | mg |
| Water | 0.1 | mL | 0.1 | mL |
| PEG-400 | 0.9 | mL | 0.9 | mL |

TABLE 19
Stability data of Formulations 40013-118 and 40013-144

| | 40013-118, | 40013-144, |
|---|---|---|
| | 3 months @ | 3 months @ |
| Parameters | 40° C./75% RH | 40° C./75% RH |
| Assay | 90.0 | 96.50 |
| pH of solution | 3.196 | 2.90 |
| (1:10 dilution with | | |
| water), Time-T0 | | |

Organic impurities, RRT (%)

| | | |
|---|---|---|
| 0.17 | 0.23 | 0.36 |
| 0.20 | 0.07 | 0.08 |
| 0.28 | 0.07 | — |
| 0.29 | 0.22 | 0.06 |
| 0.41 | 1.17 | 1.26 |
| 0.529 | — | 1.26 |
| 0.83 | — | 0.04 |
| 0.87 | — | 0.09 |
| 0.91 | 0.16 | 0.17 |
| 0.94 | 0.18 | 0.19 |
| 0.97 | 0.10 | 0.15 |
| 1.02 | 0.09 | — |
| 1.05 | 0.08 | — |
| 1.11 | 0.17 | 0.08 |
| 1.126 | 0.07 | 0.11 |
| 1.205 | — | — |
| 1.220 | — | — |
| 1.28 | 0.09 | 0.20 |
| 1.45 | 0.21 | 0.50 |
| 1.52 | 2.44 | 1.19 |
| 1.57 | — | 0.06 |
| 1.59 | 0.05 | 0.08 |
| 1.60 | 0.06 | 0.09 |
| 1.62 | 0.05 | 0.09 |
| 1.63 | 0.04 | 0.04 |
| 1.64 | 0.02 | 0.01 |
| Total impurities (%) >0.1% | 4.66 | 4.02 |
| Total impurities (%), All | 5.86 | 4.74 |

The stability of Formulations 40013-118 and 40013-144 was determined by HPLC, with conditions of 40° C./75% RH at 3 months (Table 19). Formulations with the antioxidant combinations of BHA/MTG showed better stability in terms of impurities and assay. Also, increasing BHA concentration resulted in enhanced stability.

### Example 6: Effect of Alkalinity on Formulation Containing BHA-MTG

Formulations 40013-153 and 40013-154 (Table 20) were prepared by first dissolving NaOH, MagCl₂, ETDA, HPβCD in water and BHA, MTG were dissolved in PEG-400. Both water solution and PEG-400 solutions were mixed and finally melphalan HCl was added to the mixture and sonicated until a clear solution was formed. The stability of Formulations 40013-153 and 40013-154 was determined by HPLC, with conditions of 40° C./75% RH at 3 months (Table 21).

TABLE 20
Composition for Formulations 40013-153 and 40013-154

| | 40013-153 | 40013-154 |
|---|---|---|
| Ingredients | Qty/mL | Qty/Batch |
| Melphalan HCl | 25 | 25 |
| HPβCD | 50 | 50 |
| EDTA Na2 | 0.1 | 0.1 |
| NaOH | 0.4 | 0.5 |
| BHA | 0.4 | 0.4 |
| MgCl₂ | 10 | 10 |
| MTG | 2.5 | 2.5 |
| MilliQ water | 0.1 mL | 0.1 mL |
| PEG-400 | q.s. to 1 mL | q.s. to 1 mL |

TABLE 21
Stability data for Formulations 40013-153 and 40013-154

| | 40013-153, | 40013-154, |
|---|---|---|
| | 3 months @ | 3 months @ |
| Parameters | 40° C./75% RH | 40° C./75% RH |
| Assay | 94.5 | 92.4 |
| pH of solution | 2.54 | 2.61 |
| (1:10 with water), Time-T0 | | |

Organic impurities, RRT (%)

| | | |
|---|---|---|
| 0.18 | 0.15 | 0.14 |
| 0.42 | 1.09 | 1.10 |
| 0.91 | 0.15 | 0.15 |
| 0.94 | 0.17 | 0.17 |
| 0.97 | 0.16 | 0.16 |
| 1.44 | 0.09 | 0.14 |
| 1.51 | 0.91 | 1.12 |
| 1.55 | 0.17 | 0.16 |
| 1.57 | 0.19 | 0.18 |
| 1.58 | 0.2 | 0.18 |
| 1.59 | 0.16 | 0.13 |
| 1.61 | 0.1 | 0.08 |
| Total impurities, % (>0.1%) | 3.45 | 3.63 |
| Total impurities, % (All) | 3.94 | 4.27 |

The comparative data (Table 21) of Formulations 40013-153 and 40013-154 with 0.4 and 0.5 mg/mL of NaOH, respectively, indicate that Formulation 40013-153 with a lower amount of NaOH was better in terms of stability. The small differences in the amount of NaOH, of 0.1 mg, and resulting pH difference, 0.07 units, lead to significant less total impurities, 0.33%.

Formulation 40013-144 with more BHA (1 mg/ml) from Example 5 showed more degradation (Table 19) compared to Formulation 40013-153 with less BHA (0.4 mg/mL of BHA). This could be attributed to a higher amount of melphalan HCl in Formulation 40013-153. The higher amount of melphalan HCl brought down the pH, Formulation 40013-153 has a pH of 2.54 vs 2.90 for Formulation 40013-144. Hence, pH of the formulation greatly influences the stability.

### Example 7: Formulations with BHA Containing 5% Water

Formulation 40013-160 was prepared (Table 22) by first dissolving NaOH, MagCl₂, ETDA, HPβCD in water and BHA, TPGS were dissolved in PEG-400. Both water solution and PEG-400 solutions were mixed and finally melphalan HCl was added to the mixture and sonicated until a clear solution was formed. Formulation 40013-161 was prepared (Table 22) in the same manner previously described, except MTG was used in replace of TPGS. The stability of Formulations 40013-160 and 40013-161 was determined by HPLC, with conditions of 40° C./75% RH at 2 months and 40° C./75% RH at 3 months, respectively (Table 23).

TABLE 22
Composition for Formulations 40013-160 and 40013-161

| | | |
|---|---|---|
| Ingredients | 40013-160 Qty/mL | 40013-161 Qty/mL |

| | | | | |
|---|---|---|---|---|
| Melphalan HCl | 25 | mg | 25 | mg |
| HPβCD | 50 | mg | 50 | mg |
| NaOH | 0.4 | mg | 0.4 | mg |
| EDTA Na | 0.1 | mg | 0.1 | mg |
| BHA | 1.0 | mg | 1.0 | mg |
| Vit. E TPGS | 2.5 | mg | 0.0 | |

| | | | |
|---|---|---|---|
| MTG | 0.0 | 2.5 | mg |

| | | | | |
|---|---|---|---|---|
| MgCl₂ | 10 | mg | 10 | mg |
| Purified Water | 0.05 | mL | 0.05 | mL |
| PEG-400 | 0.95 | mL | 0.95 | mL |

TABLE 23
Stability data of Formulations 40013-160 and 40013-161

| | 40013-160, | 40013-161, |
|---|---|---|
| | 2 months @ | 3 months @ |
| Parameters | 40° C./75% RH | 40° C./75% RH |
| Assay (%) | 93.5 | 96.0 |

Organic impurities, RRT (%)

| | | |
|---|---|---|
| 0.19 | 0.0 | 0.18 |
| 0.30 | 0.11 | 0.0 |
| 0.43 | 0.20 | 0.27 |
| 1.50 | 0.23 | 0.34 |
| 1.52 | 0.08 | 0.13 |
| 1.55 | 0.14 | 0.19 |
| 1.56 | 0.16 | 0.21 |
| 1.58 | 0.16 | 0.19 |
| 1.59 | 0.12 | 0.15 |
| 1.60 | 0.08 | 0.09 |
| 1.61 | 0.04 | 0.04 |
| 1.62 | 0.02 | 0.02 |
| 1.75 | 0.16 | 0.02 |
| Total impurities, >0.1% | 1.68 | 1.66 |
| (%) | | |
| Total impurities, All (%) | 2.16 | 2.37 |

Reducing water to 5% v/v in Formulations 40013-160 and 40013-161 resulted in enhanced stability (Table 23). Both the increase of BHA and the reduction in water resulted in significant improvement in stability (Table 23). Formulation 40013-161 exhibited the lowest impurity percentage (2.37%) at 40° C./75% RH after 3 months compared to any other formulations tested at these same conditions (Formulations 40013-144, 40013-153, and 40013-154).

The combination of BHA-MTG worked better than BHA-TPGS. Formulation 40013-160 has the same amount of impurities at 2 months compared to the 3 months data at 40° C./75% RH (Table 23). This comparative result suggests that the BHA-MTG combination worked relatively better than BHA-TPGS, since 2 months data of Formulation 40013-160 is equal to three months data of Formulation 40013-161.

### Example 8: Formulations with BHA-MTG with Different Concentration of EDTA

Formulations 40013-161 and 40013-162B were prepared (Table 24) by first dissolving NaOH, MagCl₂, ETDA, HPβCD in water and BHA, MTG was dissolved in PEG-400. Both water solution and PEG-400 solutions were mixed and finally melphalan HCl was added to the mixture and sonicated until a clear solution was formed. The stability of Formulations 40013-161 and 40013-162B was determined by HPLC, with conditions of 40° C./75% RH at 4 months (Table 25).

TABLE 24
Composition for Formulations 40013-161 and 40013-162B

| | | |
|---|---|---|
| Ingredients | 40013-161 Qty/mL | 40013-162B Qty/mL |

| | | | | |
|---|---|---|---|---|
| Melphalan HCl | 25 | mg | 25 | mg |
| HPβCD | 50 | mg | 50 | mg |
| NaOH | 0.4 | mg | 0.4 | mg |
| EDTA Na | 0.1 | mg | 0.05 | mg |
| BHA | 1.0 | mg | 1.0 | mg |
| MTG | 2.5 | mg | 2.5 | mg |
| MgCl₂ | 10 | mg | 10 | mg |
| Purified Water | 0.05 | mL | 0.05 | mL |
| PEG-400 | 0.95 | mL | 0.95 | mL |

TABLE 25
Comparative Stability Results for Formulations
40013-161 and 40013-162B

| | 40013-161, | 40013-162B, |
|---|---|---|
| | 6 months @ | 6 months @ |
| | 40° C./75% RH | 40° C./75% RH |
| Parameter | % Impurity | % Impurity |
| Assay | 93.3 | 93.3 |

Organic impurities, RRT (%)

| | | |
|---|---|---|
| Impurity D | 0.45 | 0.45 |
| Impurity G | 0.58 | 0.66 |
| Impurity H | 0.45 | 0.29 |
| 0.19 | 0.15 | 0.22 |
| 0.91 | 0.13 | 0.14 |
| 0.94 | 0.16 | 0.16 |
| 0.97 | 0.14 | 0.15 |
| 1.11 | <0.1 | <0.1 |
| 1.27 | <0.1 | <0.1 |
| 1.34 | <0.1 | <0.1 |
| 1.44 | <0.1 | 0.13 |
| 1.56 | 0.30 | 0.47 |
| 1.58 | 0.52 | 0.54 |
| 1.59 | 0.50 | 0.52 |
| 1.60 | 0.39 | 0.40 |
| 1.61 | 0.24 | 0.25 |
| 1.62 | 0.13 | 0.13 |
| Total impurities % (>0.1) | 4.29 | 4.51 |
| Total impurities % (All) | 4.75 | 5.07 |

Decreasing the EDTA concentration to 0.05 from 0.1 mg/mL resulted in a moderate increase in impurities by about 0.3%. Hence, the EDTA at 0.10 mg/mL looked optimum as Formulation 40013-162B with 0.05 mg/mL of EDTA had slightly more impurities.

### CONCLUSIONS

In summary, melphalan has limited stability in organic solvents even with antioxidants. Surprisingly, the addition of water had a positive effect on stability. The inclusion of EDTA improved the stability even more significantly, with impurities reduced to half (e.g., Formulations F-12 vs F-51).

The chloride ion source had a positive effect. Magnesium chloride was more effective in stabilizing the product than NaCl.

The combination of MTG-TPGS-MgCl₂ stabilized the melphalan and was suitable for refrigerator storage. However, haziness appeared after refrigeration and required 60 minutes after thawing to attain clarity.

The inclusion of cysteine resulted in enhanced chemical stability but physical stability was an issue as precipitation occurred after a couple of months. The lower pH of around 2 was needed but it is physiologically incompatible.

BHA-MTG or BHA-TPGS offered greater stability, but BHA-MTG showed better stabilizing effects than BHA-TPGS. And water at around 5% v/v resulted in greater stability.

Finally, an EDTA concentration of 0.10 mg/mL showed better stabilizing effects.

1. A stable, liquid pharmaceutical composition comprising:
a) melphalan;
b) at least one cyclodextrin;
c) at least one non-aqueous solvent;
d) water and/or at least one aqueous buffer;
e) at least one antioxidant;
f) optionally, at least one chelating agent; and
g) optionally, at least one inorganic salt.

2. The composition of claim 1, wherein the melphalan is present in the composition in an amount ranging from 1-100 mg/mL.

3. The composition of claim 1, wherein the cyclodextrin is selected from the group consisting of hydroxy propyl-β-cyclodextrin, hydroxy propyl-γ-cyclodextrin, and mixtures thereof.

4. (canceled)

5. The composition of claim 1, wherein the composition does not contain a charged cyclodextrin and/or a sulfoalkyl ether cyclodextrin, or derivative thereof.

6. The composition of claim 1, wherein the at least one cyclodextrin is present in the composition in an amount ranging from about 1-20% w/v.

7. The composition of claim 1, wherein the non-aqueous solvent is selected from the group consisting of PEG 300, 400, PEG 600, and mixtures thereof.

8. (canceled)

9. (canceled)

10. The composition of claim 1, wherein the water and/or at least one aqueous buffer is present in the composition in an amount of up to about 40% v/v.

11. (canceled)

12. (canceled)

13. The composition of claim 1, wherein the antioxidant is selected from the group consisting of monothioglycerol (MTG), cysteine, tocopherol or derivative thereof, butylated hydroxyl anisole (BHA), butylated hydroxyl toluene (BHT), inorganic sulfates, aromatic compounds, and mixtures thereof.

14. (canceled)

15. The composition of claim 1, wherein the antioxidant is present in the composition in an amount ranging from about 0.04-0.5% w/v.

16. The composition of claim 1, wherein the chelating agent is ethylenediaminetetraacetic acid (EDTA) or its salts.

17. (canceled)

18. The composition of claim 1, wherein the chelating agent is present in the composition in an amount ranging from about 0.01-0.5 mg/mL.

19. The composition of claim 1, wherein the inorganic salt is a chloride salt.

20. (canceled)

21. (canceled)

22. The composition of claim 1, wherein the inorganic salt is present in the composition in an amount ranging from about 0.01-5 wt %.

23. The composition of claim 1, further comprising at least one pharmaceutically acceptable excipient.

24. The composition of claim 23, wherein the pharmaceutically acceptable excipient is selected from the group consisting of at least one surfactant, at least one antimicrobial, at least one preservative, at least one alkalizer and pH modifying agent, and mixtures thereof.

25. (canceled)

26. The composition of claim 24, wherein the pharmaceutically acceptable excipient is present in the composition in an amount of about 0.01-2% v/v.

27. The composition of claim 1, wherein:
the melphalan is present in the composition in an amount ranging from 1-100 mg/mL;
the at least one cyclodextrin is selected from the group consisting of hydroxy propyl-β-cyclodextrin, hydroxy propyl-γ-cyclodextrin, and mixtures thereof;
the at least one non-aqueous solvent is selected from the group consisting of PEG 300, 400, PEG 600, and mixtures thereof;
water is present;
the at least one antioxidant is selected from the group consisting of monothioglycerol (MTG), cysteine, tocopherol or derivative thereof, butylated hydroxyl anisole (BHA), butylated hydroxyl toluene (BHT), inorganic sulfates, aromatic compounds, and mixtures thereof;
the at least one chelating agent is EDTA or its salts; and
the at least one inorganic salt is a chloride salt,
wherein the composition further comprises an alkalizer selected from sodium hydroxide.

28. (canceled)

29. (canceled)

30. (canceled)

31. (canceled)

32. The composition of claim 27, wherein the composition comprises:
about 1-5 wt % of the at least one cyclodextrin;
about 30-95% v/v of the at least one non-aqueous solvent;
about 5% v/v of the water;
about 0.04-0.5 wt % of the at least one antioxidant;
about 0.1 mg/mL of the at least one chelating agent;
about 1-2 wt % of the at least one inorganic salt; and
about 1.5-2 wt % of the alkalizer.

33. The composition of claim 27, wherein the composition does not contain a charged cyclodextrin and/or a sulfoalkyl ether cyclodextrin or derivative thereof.

34. The composition of claim 1, wherein the composition contains total impurities ≤6% in the composition resulting from the degradation of melphalan in the composition, as determined by HPLC at a wavelength of 260 nm, at about 2-8° C. for ≥1 years.

35. A sealed unit dose of the composition of claim 1.

36. A method of treating cancer comprising the administration of an effective amount of the composition of claim 1 to a mammal in need thereof.

37. (canceled)

38. (canceled)

39. (canceled)

40. (canceled)

41. (canceled)
